(19) United States
(12) Patent Application Publication
(10) Pub. No.: US 2023/0270798 A1
(43) Pub. Date: Aug. 31, 2023

(54) SYNBIOTIC COMPOSITION

(71) Applicant: SOCIETE DES PRODUITS NESTLE S.A., Vevey (CH)

(72) Inventors: BILJANA BOGICEVIC, Bern (CH); GUENOLEE ELIANE MARIE PRIOULT, Bern (CH)

(21) Appl. No.: 18/003,169

(22) PCT Filed: Jun. 25, 2021
(86) PCT No.: PCT/EP2021/067447

§ 371 Date: Dec. 23, 2022

(30) Foreign Application Priority Data
Jun. 26, 2020 (EP) 20182596.5

(51) Int. Cl. A61K 35/745; A23L 33/135; A23L 33/00; A61K 31/702; A61P 1/00

(52) U.S. Cl. CPC A61K 35/745; A23L 33/135; A23L 33/40; A61K 31/702; A61P 1/00

(57) ABSTRACT

The present invention relates to a composition or to a kit of compositions comprising Bifidobacterium longum subsp longum and Lacto-N-tetraose (LNT) for use in increasing microbiota activity, for improving engraftment and/or survival of Bifidobacteria, such as B. longum subsp. longum, for promoting the growth of beneficial bacteria in the gut, such as for promoting the growth of Streptococcus salivarius, for treating and/or preventing inflammatory conditions in the gut, for preventing and/or treating infections, for improving immunity and/or for improving oral health.

### FIELD OF THE INVENTION

The present invention relates to a composition or to a kit of compositions comprising Bifidobacterium longum subsp. longum and Lacto-N-tetraose (LNT) for use in increasing microbiota activity, for improving engraftment and/or survival of Bifidobacteria, such as B. longum subsp. longum, for promoting the growth of beneficial bacteria in the gut, such as for promoting the growth of Streptococcus salivarius, for treating and/or preventing inflammatory conditions in the gut, for preventing and/or treating infections, for improving immunity and/or for improving oral health.

### BACKGROUND TO THE INVENTION

In healthy, vaginally-delivered, breast-fed infant, Bifidobacteria form the basis of the microbiota in the infant gut. The amount of Bifidobacteria varies over the first months of life starting in average slightly below 10% at birth and increasing up to a peak of nearly 40% between 2.5 and 3 months. Breast feeding promotes intestinal barrier development which, together with bifidobacterial domination leads to enhanced absorption and therefore utilisation of ingested nutrition, enhanced protection against infections and proper modulation of immunity thus reducing infections and inflammation.

According to recent estimates and depending on the geographical location, B. longum subsp. longum can make up to 20% of the Bifidobacterium community in the intestine. At the same time, genomic studies have convincingly shown that Bifidobacteria present in the gut of breast-fed infants, such as Bifidobacterium longum, are specially equipped to utilize breast-milk oligosaccharides as nutrients. Bifidobacterium longum is also adapted to the conditions in the large intestine where energy harvest from slowly absorbable carbohydrates takes place (Turroni et al. 2018, Bifidobacteria and the infant gut: an example of co-evolution and natural selection. Cell 390 Mol Life Sci 75:103-118).

More and more evidence is emerging which suggests that the establishment of an appropriate intestinal microbiota early in life, including population of B. longum subsp. longum, may be significant in subsequent healthy development. It is therefore clear that there is a need to provide a means to promote the rapid establishment of an appropriate intestinal microbiota in infants.

Additionally, there is a need to provide nutritional compositions which could improve microbiota activity, improve engraftment and/or survival of Bifidobacteria, in particular of B. longum subsp. longum, to promote the growth of beneficial bacteria in the gut, which in turn positively impacts treatment and/or prevention of inflammatory conditions in the gut, prevention and/or treatment of infections, improvement of immune response and/or improvement of oral health in infants and young children.

### SUMMARY OF THE INVENTION

The present inventors have surprisingly found that a synergistic effect is obtained when LNT is used in combination with Bifidobacterium longum subsp. longum in a nutritional composition for infants and young children. The combination of the probiotic B. longum subsp. longum with the LNT synergistically promotes a healthier gut environment thanks to the enhancement of beneficial fermentative metabolites such as short chain fatty acids, in particular acetate and the growth stimulation of healthy commensal gut bacteria such as Streptococcus salivarus, known namely for its anti-inflammatory properties.

Accordingly, in a first aspect of the present invention, there is provided a nutritional composition comprising LNT and B. longum subsp. longum or a kit of nutritional compositions comprising a first composition comprising B. longum subsp. longum and a second composition comprising LNT, for use in
increasing the metabolic activity of the gut microbiota of the subject consuming the composition, preferably increasing the production of fermentation metabolites, more preferably increasing the production of short chain fatty acids, most preferably increasing the production of acetate and/or propionate; preferably these benefits are achieved in the colon of the subject consuming the composition;
reducing the production of ammonium in the gut, preferably in the colon of the subject consuming the composition;
improving engraftment and/or survival of Bifidobacteria, preferably of Bifidobacterium longum subsp. longum in the colon of the subject consuming the composition;
promoting the growth and/or the survival of beneficial bacteria in the gut and/or the oral cavity of the subject consuming the composition, preferably promoting the growth and/or the survival of Streptococcus salivarius in the gut and/or in the oral cavity, preferably in the colon and/or the oral cavity of the subject consuming the composition;
promoting the growth and/or survival of propionate-producing microorganism in the gut, preferably in the colon of the subject consuming the composition;
treating, preventing and/or reducing inflammation in the subject consuming the composition, preferably inflammatory conditions in the gut and/or in the oral cavity, more preferably in the colon of the subject consuming the composition;
treating, preventing and/or reducing infections, such as bacterial infections, preferably gastrointestinal and/or oral infections (such as pharyngeal infection, for example Streptococcus pyogenes infection) in the subject consuming the composition;
improving immunity in the subject consuming the composition
preventing caries in the subject consuming the composition.
   wherein the subject is an infant (up to 12 months of age) or a young child aged from 1 to 3 years of age).

In a second aspect there is provided a growing-up milk comprising LNT and B. longum subsp. longum, for use in
increasing the metabolic activity of the gut microbiota of the subject consuming the composition, preferably increasing the production of fermentation metabolites, more preferably increasing the production of short chain fatty acids, most preferably increasing the production of acetate and/or propionate; preferably these benefits are achieved in the colon of the subject consuming the composition;
reducing the production of ammonium in the gut, preferably in the colon of the subject consuming the composition;
improving engraftment and/or survival of Bifidobacteria, preferably of Bifidobacterium longum subsp. longum in the colon of the subject consuming the composition;
promoting the growth and/or the survival of beneficial bacteria in the gut and/or the oral cavity of the subject consuming the composition, preferably promoting the growth and/or the survival of Streptococcus salivarius in the gut and/or in the oral cavity, preferably in the colon and/or the oral cavity of the subject consuming the composition;
promoting the growth and/or survival of propionate-producing microorganism in the gut, preferably in the colon of the subject consuming the composition;
treating, preventing and/or reducing inflammation in the subject consuming the composition, preferably inflammatory conditions in the gut and/or in the oral cavity, more preferably in the colon of the subject consuming the composition;
treating, preventing and/or reducing infections, such as bacterial infections, preferably gastrointestinal and/or oral infections (such as pharyngeal infection, for example Streptococcus pyogenes infection) in the subject consuming the composition;
improving immunity in the subject consuming the composition
preventing caries in the subject consuming the composition.
   wherein the subject is a child above 3 years and below 8 years of age).

### DESCRIPTION OF THE DRAWINGS

FIG. 1—Total short chain fatty acid concentration after incubation for 24 hours with the blank, the probiotic incubation (Bifidobacterium longum subsp. longum ATCC BAA-999 alone), the prebiotic incubation (LNT alone) and the synbiotic incubation (Bifidobacterium longum subsp. longum ATCC BAA-999 and LNT).

FIG. 2—Acetate concentration after incubation for 24 hours with the blank, the probiotic incubation (Bifidobacterium longum subsp. longum ATCC BAA-999 alone), the prebiotic incubation (LNT alone) and the synbiotic incubation (Bifidobacterium longum subsp. longum ATCC BAA-999 and LNT).

FIG. 3—Propionate concentration after incubation for 24 hours with the blank, the probiotic incubation (Bifidobacterium longum subsp. longum ATCC BAA-999 alone), the prebiotic incubation (LNT alone) and the synbiotic incubation (Bifidobacterium longum subsp. longum ATCC BAA-999 and LNT).

FIG. 4—Ammonium concentration after incubation for 48 hours with the blank, the probiotic incubation (Bifidobacterium longum subsp. longum ATCC BAA-999 alone), the prebiotic incubation (LNT alone) and the synbiotic incubation (Bifidobacterium longum subsp. longum ATCC BAA-999 and LNT).

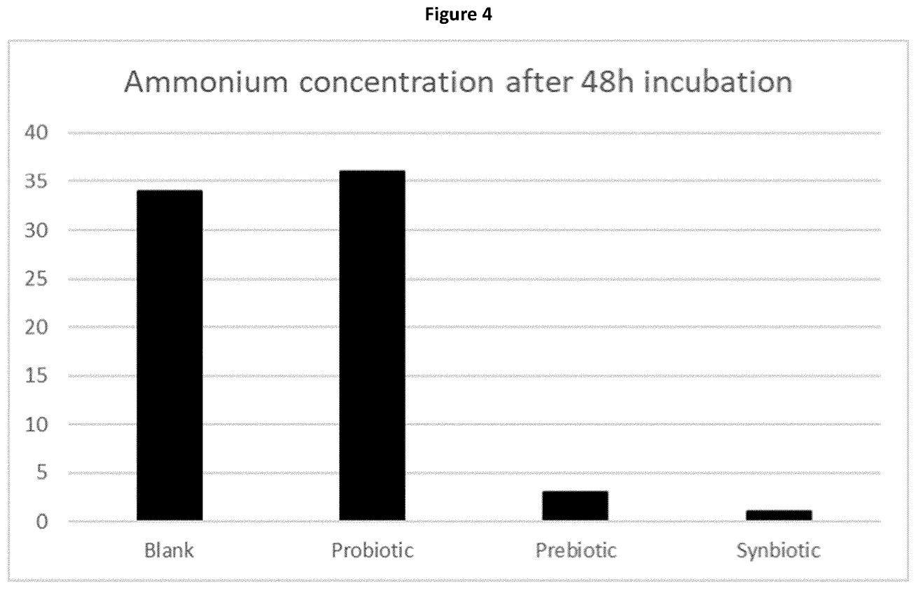

FIG. 5—Population of B. longum subsp. longum after incubation for 6 and 48 hours with the blank, the probiotic incubation (Bifidobacterium longum subsp. longum ATCC BAA-999 alone), the prebiotic incubation (LNT alone) and the synbiotic incubation (Bifidobacterium longum subsp. longum ATCC BAA-999 and LNT).

FIG. 6—Population of Streptococcus salivarius after incubation for 6 and 48 hours with the blank, the probiotic incubation (Bifidobacterium longum subsp. longum ATCC BAA-999 alone), the prebiotic incubation (LNT alone) and the synbiotic incubation (Bifidobacterium longum subsp. longum ATCC BAA-999 and LNT).

### DETAILED DESCRIPTION OF THE INVENTION

### Definitions

Within the context of the present invention the term “bifidogenic intestinal microbiota” means an intestinal microbiota which is dominated by Bifidobacteria such as Bifidobacterium breve, Bifidobacterium infantis, Bifidobacterium bifidum and Bifidobacterium longum.

The term “infant” means a child under the age of 12 months. The expression “child” means a between 12 months and seven years of age. The expression “young child” means a child aged between one and less than three years, also called toddler.

An “infant or young child born by C-section” means an infant or young child who was delivered by caesarean. It means that the infant or young child was not vaginally delivered.

An “infant or young child vaginally born” means an infant or young child who was vaginally delivered and not delivered by caesarean.

A “preterm” or “premature” means an infant or young child who was not born at term. Generally it refers to an infant or young child born prior 37 weeks of gestation.

An “infant having a low birth weight” means a new born having a body weight below 2500 g (5.5 pounds) either because of preterm birth or restricted foetal growth. It therefore encompasses:
infant or young child who has/had a body weight from 1500 to 2500 g at birth (usually called “low birth weight” or LBW)
infant or young child who has/had a body weight from 1000 to 1500 g at birth (called “very low birth weight” or VLBW)
infant or young child who has/had a body weight under 1000 g at birth (called “extremely low birth weight” or ELBW).

An “infant born small for gestational age (SGA)” means a baby with birth weights below the 10^{th} percentile for babies of the same gestational age.

The expression “nutritional composition” means a composition which may nourish a subject. This nutritional composition is usually to be taken orally or intravenously, and it usually includes a lipid or fat source, a carbohydrate source and/or and a protein source. Non limiting examples of nutritional compositions are: infant formula, follow up formula, baby food, growing up milk, fortifier, paediatric supplements or infant cereal compositions.

In a particular embodiment the composition of the present invention is a hypoallergenic nutritional composition. The expression “hypoallergenic nutritional composition” means a nutritional composition which is unlikely to cause allergic reactions.

In a particular embodiment the composition or nutritional composition of the present invention is a “synthetic nutritional composition”. The expression “synthetic nutritional composition” means a mixture obtained by chemical and/or biological means, which can be chemically identical to the mixture naturally occurring in mammalian milks (i.e. the synthetic composition is not breast milk).

The expression “infant formula” as used herein refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The expression “growing-up milk” (or GUM) refers to a milk-based drink generally with added vitamins and minerals, that is intended for young children or children.

The term “fortifier” refers to liquid or solid nutritional compositions suitable for fortifying or mixing with human milk, infant formula, growing-up milk or human breast milk fortified with other nutrients. Accordingly, the fortifier of the present invention can be administered after dissolution in human breast milk, in infant formula, in growing-up milk or in human breast milk fortified with other nutrients or otherwise it can be administered as a stand-alone composition. When administered as a stand-alone composition, the milk fortifier of the present invention can be also identified as being a “supplement”. In one embodiment, the milk fortifier of the present invention is a supplement.

The term “nutritional supplement” refers to a product which is intended to supplement the general diet of a subject.

The expression “weaning period” means the period during which the mother's milk is substituted by other food in the diet of an infant or young child.

The expressions “gastrointestinal tract”, “GI tract”, “GIT”, “gut” and “GUT” can be used interchangeably. The tract consists of the stomach and intestines, and is divided into the upper gastrointestinal tract and the lower gastrointestinal tract. It refers to the system (including digestive organs) responsible for consuming and digesting foodstuffs, absorbing nutrients, and expelling waste. The GI tract especially includes all digestive structures between the mouth and the anus. The upper gastrointestinal tract typically includes the oesophagus and the stomach. The lower gastrointestinal tract typically includes the small intestine and all of the large intestine (colon).

The expression “preventing and/or treating gastrointestinal inflammations” encompasses one or several of the following:
preventing gastrointestinal inflammations, i.e. inflammations of the gut
treating gastrointestinal inflammations, i.e. inflammations of the gut

### Composition

The composition or the kit of compositions for use according to the present invention comprises at least one strain of Bifidobacterium longum subsp. longum and the human milk oligosaccharide Lacto-N-tetraose (LNT). Suitable sources of LNT are commercially available and include for example Glycom A/S in Denmark.

In one embodiment, the invention refers to a composition comprising both Bifidobacterium longum subsp. longum and LNT in admixture. In another embodiment, the Bifidobacterium longum subsp.

longum and LNT, are present in the form of a kit of compositions comprising a preparation of the two agents and, optionally, instructions for the simultaneous or sequential administration of the preparations to a subject in need thereof. In a preferred aspect, the invention refers to a composition wherein Bifidobacterium longum subsp. longum and LNT are in admixture.

The Bifidobacterium longum subsp. longum may be any Bifidobacterium longum subsp. longum strain. In some preferred embodiments the Bifidobacterium longum subsp. longum strain is selected from Bifidobacterium longum subsp. longum ATCC BAA-999 sold by Morinaga Milk Industry Co. Ltd. of Japan under the trademark BB536, Bifidobacterium longum subsp. longum strain CNCM I-2169, Bifidobacterium longum subsp. longum strain CNCM I-2171, Bifidobacterium longum subsp. longum strain ATCC 15708, Bifidobacterium longum subsp. longum strain DSM 20097, Bifidobacterium longum subsp. longum strain NCIMB 8809, Bifidobacterium longum subsp. longum strain CNCM I-2618 (NCC 2705), Bifidobacterium longum subsp. longum strain CNCM I-2170, Bifidobacterium longum subsp. longum strain ATCC 15707 (T), Bifidobacterium 30 longum subsp. longum strain CNCM I-103, Bifidobacterium longum subsp. longum strain CNCM I-2334, Bifidobacterium longum subsp longum strain CNCM I-3864, Bifidobacterium longum subsp longum strain CNCM I-3853, or a combination thereof.

In a preferred embodiment, the Bifidobacterium longum subsp longum strain is selected from Bifidobacterium longum subsp. longum ATCC BAA-999, Bifidobacterium longum subsp. longum strain CNCM I-2169, Bifidobacterium longum subsp. longum strain CNCM I-2171, Bifidobacterium longum subsp. longum strain ATCC 15708, Bifidobacterium longum subsp. longum strain DSM 20097, Bifidobacterium longum subsp. longum strain NCIMB 8809, Bifidobacterium longum subsp. longum strain NCIMB 8810, Bifidobacterium longum subsp. longum strain CNCM I-2618 (NCC 2705), Bifidobacterium longum subsp. longum strain ATCC 15707 (T), or a combination thereof.

In a particularly preferred embodiment, the Bifidobacterium longum subsp. longum strain is B. longum subsp. longum ATCC BAA-999 or B. longum subsp. longum CNCM I-2618 (NCC 2705), most preferably, it is B. longum subsp. longum ATCC BAA-999.

The strains have been deposited in the depositary institution indicated in the table below (Table 1), and have received the following date of deposit and accession number:

TABLE 1

| | Depositary | Accession | |
|---|---|---|---|
| # | institution | number | Date of deposit |
| 1 | CNCM | I-2618 | 29 Jan. 2001 |
| 2 | ATCC | 15707 | <1990 |
| 3 | CNCM | I-2169 | 15 Mar. 1999 |
| 4 | NCIMB | 8810 | 1 Oct. 1956 |
| 5 | CNCM | I-2171 | 15 Feb. 1999 |
| 6 | ATCC | 15708 | <1990 |
| 7 | DSM | 20097 | <1990 |
| 8 | NCIMB | 8809 | 1 Oct. 1956 |
| 9 | CNCM | I-2170 | 15 Mar. 1999 |
| 10 | CNCM | I-103 | 29 Mar. 1991 |
| 11 | CNCM | I-2334 | 12 Oct. 1999 |
| 12 | CNCM | I-3864 | 15 Nov. 2007 |
| 13 | CNCM | I-3853 | 16 Oct. 2007 |

CNCM refers to Collection nationale de cultures de micro-organismes, Institut Pasteur, 28, rue du Dr Roux, F-75724 Paris Cedex 15, France. ATCC refers to American Type Culture Collection 10801 University Blvd., Manassas, Va. 20110-2209, U.S.A. DSM refers to Leibniz Institute DSMZ-German Collection of Microorganisms and Cell Cultures, Inhoffenstr. 7B, D-38124 Braunschweig, Germany. NCIMB refers to NCIMB Ltd, Ferguson Building, Craibstone Estate, Buckburn, Aberdeen AB21 9YA, Scotland.

Strains 1, 2, 5, 9-13 have been deposited by Nestec S. A., avenue Nestlé 55, 1800 Vevey, Switzerland. Since then, Nestec S. A. has merged into Société des Produits Nestlé S. A. Accordingly, Société des Produits Nestlé S. A. is the successor in title of Nestec S. A., under article 2(ix) of the Budapest Treaty. All other strains are commercially available.

The B. longum subsp. longum can be produced using any method known in the art and using any suitable medium. A non-limiting example of a typical growth medium for B. longum is MRS (De Man, Rogosa and Sharpe) medium, optionally a MRS medium from which carbon source has been removed or an MRS medium supplemented with 0.05% of cysteine (MRSc).

In a particular aspect, the B. longum subsp. longum can be advantageously produced by a method of growing the B. longum subsp. longum in a culture medium comprising LNT. This is believed to boost the synergy between the B. longum subsp. longum strain and LNT by preconditioning the strain for consumption of LNT when the strain is faced again with LNT in the gastrointestinal tract of the subject consuming the composition. Preferably the growth medium comprises LNT in a concentration of 0.02 to 5 wt %, 0.05 to 2 wt %, 0.1 to 1.5 wt %, or about 0.5%.

LNT may be added to a conventional culture medium comprising up to 8 wt %, preferably up to 6 wt %, for example up to 4 wt %, of another sugar suitable to sustain B. longum growth, such as, but not limited to, glucose. Preferably the culture medium at the end of the fermentation contains less than 0.4 wt % glucose, such as from 0 wt % to 0.3 wt % glucose, for example from 0.02 wt % to 0.4 wt %, or from about 0.05 wt % to about 0.3 wt %. Conventional culture mediums suitable for growth of B. longum are well known to the person skilled in the art.

Strains belonging to the species B. longum are grown in anaerobic conditions. Fermentation methods under anaerobic conditions are commonly known. The skilled person is able to identify suitable components of the fermentation medium and to adjust fermentation conditions based on his general knowledge, depending on the microorganism to be grown. The fermentation medium typically comprises a nitrogen source such as yeast extract, a carbon source such as a sugar, various growth factors (e.g. minerals, vitamins etc.) required by the microorganism and water.

The fermentation is preferably carried out in two steps, a starter fermentation being carried out prior to the main fermentation step. The fermentation medium can be different for the starter and the main fermentation or may be identical.

The second step of the process is the concentration of the biomass. This can also be carried out using methods known to the person skilled in the art, such as for example centrifugation or filtration. The total solid content of the biomass after concentration is preferably comprised between 10 and 35 wt %, preferably between 14 and 35 wt %, based on the total dry weight of the biomass (i.e. of the total amount of fermentation medium and produced microorganism). Optionally, the concentration may be preceded or combined with a washing step to remove residues of the fermentation medium and/or compounds produced during fermentation. For example, washing may be performed by concentrating biomass, re-suspending the concentrated biomass in a buffer, such as a phosphate buffer, or a similar composition and re-concentrating the biomass.

The nutritional composition of the present invention can be in solid form (e.g. powder) or in liquid form. The amount of the various ingredients (e.g. the oligosaccharides) can be expressed in g/100 g of composition on a dry weight basis when it is in a solid form, e.g. a powder, or as a concentration in g/L of the composition when it refers to a liquid form (this latter also encompasses liquid composition that may be obtained from a powder after reconstitution in a liquid such as milk, water . . . , e.g. a reconstituted infant formula or a follow-on/follow-up formula or an infant cereal product or any other formulation designed for infant nutrition).

It is clear to those skilled in the art that an ideal dose will depend on the subject to be treated, its health condition, sex, age, or weight, for example, and the route of administration. The dose to be ideally used will consequently vary but can be determined easily by those of skill in the art.

However, generally, it is preferred if the composition of the present invention comprises between 10³ to 10¹² cfu, more preferably between 10⁷ and 10¹² cfu such as between 10⁸ and 10¹⁰ cfu of Bifidobacterium longum subsp. longum per daily dose. It may also comprise between 10³ to 10¹² cfu of probiotic strain, more preferably between 10⁷ and 10¹² cfu such as between 10⁸ and 10¹⁰ cfu of Bifidobacterium longum subsp. longum per g of composition on a dry weight basis.of per g of the dry weight of the composition.

In an embodiment, LNT is present in an amount of 1-5 g/L of the composition or in an amount of 0.5 to 4 g per 100 g of the composition on a dry weight basis.

The nutritional composition according to the present invention may also comprise additional optional ingredients.

For example, the composition may container other types of oligosaccharide(s) (i.e. other than LNT) and/or at least a fiber(s) and/or at least a precursor(s) thereof. The other oligosaccharide and/or fiber and/or precursor thereof may be selected from the list comprising human milk oligosaccharides other than LNT, galacto-oligosaccharides (GOS), fructo-oligosaccharides (FOS), inulin, xylooligosaccharides (XOS), polydextrose and any combination thereof. They may be in an amount between 0 and 10% by weight of composition. In a particular embodiment, the nutritional composition can also contain at least one BMO (bovine milk derived oligosaccharide).

Suitable human milk oligosaccharides include fucosylated oligosaccharides, N-acetylated oligosaccharides, sialylated oligosaccharides and precursors thereof.

Fucosylated oligosaccharides are oligosaccharides having a fucose residue. These oligosaccharides have a neutral nature. Some examples are 2′-FL (2′-fucosyllactose), 3′-FL (3-fucosyllactose), difucosyllactose (DiFL), Lacto-difucotetraose (LDFT)), lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof.

N-acetylated oligosaccharides encompasse both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose) and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

Sialylated oligosaccharides are charged sialic acid containing oligosaccharides, i.e. oligosaccharides having a sialic acid residue. It has an acidic nature. Some examples are 3′-SL (3′ sialyllactose) and 6′-SL (6′ sialyllactose).

Precursor of human milk oligosaccharides are key compounds that intervene in the manufacture of HMO, such as sialic acid and/or fucose.

Suitable commercial products that can be used to prepare the nutritional compositions according to the invention include combinations of FOS with inulin such as the product sold by BENEO under the trademark Orafti, or polydextrose sold by Tate & Lyle under the trademark STA-LITE®.

The nutritional composition of the present invention can further comprise additional probiotic microorganisms. The probiotic microorganisms most commonly used are principally bacteria and yeasts, preferably bacteria. Examples of bacteria which can be protected by the composition of the present invention include bifidobacteria, lactobacilli, lacticaseibacilli, lactiplantibacilli, levilactobacilli, ligilactobacilli, limosilactobacilli, latilactobacilli, lactococci, enterococci, streptococci, Leuconostoc, Escherichia, propionibacteria, or combinations thereof, preferably Bifidobacterium lactis, Bifidobacterium animalis, Bifidobacterium breve, Bifidobacterium longum subsp. infantis, Bifidobacterium adolescentis, Lactobacillus acidophilus, Lacticaseibacillus casei (previously known as Lactobacillus casei), Lacticaseibacillus paracasei (previously known as Lactobacillus paracasei), Ligilactobacillus salivarius (previously known as Lactobacillus salivarius), Lacticaseibacillus rhamnosus (previously known as Lactobacillus rhamnosus), Lactobacillus johnsonii, Lactiplantibacillus plantarum (previously known as Lactobacillus plantarum), Limosilactobacillus fermentum (previously known as Lactobacillus fermentum), Lactococcus lactis, Streptococcus thermophilus, Lactococcus diacetylactis, Lactococcus cremoris, Lactobacillus delbrueckii subsp. bulgaricus, Lactobacillus delbrueckii subsp. lactis, Lactobacillus helveticus, Escherichia coli, Enterococcus faecium, Leuconostoc pseudomesenteroides, Bifidobacterium bifidum, Lactobacillus gasseri, Latilactobacillus sakei (previously known as Lactobacillus sakei), Streptococcus salivarius, and/or mixtures thereof, as well as any of their subspecies.

Examples of bacterial strains that can efficiently be protected include Bifidobacterium longum subsp. infantis (ATCC17930), Bifidobacterium breve (CNCM I-3865), Bifidobacterium lactis BL818 (CNCM I-3446), Lactobacillus johnsonii Lal (CNCM I-1225), Lacticaseibacillus paracasei (CNCM I-2116) (previously known as Lactobacillus paracasei (CNCM I-2116)), Lacticaseibacillus rhamnosus LPR (CGMCC 1.3724) (previously known as Lactobacillus rhamnosus LPR (CGMCC 1.3724)), Streptococcus thermophilus (CNCM I-1422), Streptococcus thermophilus ST496 (CNCM I-4153), Lacticaseibacillus casei (CNCM I-1518) (previously known as Lactobacillus casei (CNCM I-1518)), Lacticaseibacillus casei (ACA-DC 6002) (previously known as Lactobacillus casei (ACA-DC 6002)), Escherichia coli Nissle, Lactobacillus bulgaricus (CNCM I-1198), Lactococcus lactis (CNCM I-4154), or combinations thereof.

In one embodiment the probiotics are viable. In another embodiment the probiotics are non-replicating or inactivated. There may be both viable probiotics and inactivated probiotics in some other embodiments. Probiotic components and metabolites can also be added.

The nutritional composition according to the invention generally contains a protein source. The protein can be in an amount of from 1.6 to 3 g per 100 kcal. In some embodiments, especially when the composition is intended for premature infants, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

The type of protein is not believed to be critical to the present invention provided that the minimum requirements for essential amino acid content are met and satisfactory growth is ensured. Thus, protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some advantageous embodiments the protein source is whey predominant (i.e. more than 50% of proteins are coming from whey proteins, such as 60% or 70%).

The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids. The proteins may be either fully or partially hydrolysed. It may be desirable to supply partially hydrolysed proteins (degree of hydrolysis between 2 and 20%), for example for infants or young children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine;

for example about 7% by weight of lysine which greatly improves the nutritional quality of the protein source.

In an embodiment of the invention at least 70% of the proteins are hydrolysed, preferably at least 80% of the proteins are hydrolysed, such as at least 85% of the proteins are hydrolysed, even more preferably at least 90% of the proteins are hydrolysed, such as at least 95% of the proteins are hydrolysed, particularly at least 98% of the proteins are hydrolysed. In a particular embodiment, 100% of the proteins are hydrolysed.

In one particular embodiment the proteins of the nutritional composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 or 90.

The protein component can alternatively be replaced by a mixture or synthetic amino acid, for example for preterm or low birth weight infants.

In a particular embodiment the nutritional composition according to the invention is a hypoallergenic composition. In another particular embodiment the composition according to the invention is a hypoallergenic nutritional composition.

The nutritional composition according to the present invention generally contains a source of available carbohydrate. This is particularly preferable in the case where the nutritional composition of the invention is an infant formula. In this case, any available carbohydrate source conventionally found in infant formulae such as lactose, sucrose, saccharose, maltodextrin, starch and mixtures thereof may be used although one of the preferred sources of carbohydrates is lactose.

The nutritional composition according to the present invention generally contains a source of lipids. This is particularly relevant if the nutritional composition of the invention is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and α-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example about 8:1 to about 10:1.

The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population.

If necessary, the nutritional composition of the invention may contain emulsifiers and stabilisers such as soy, lecithin, citric acid esters of mono- and diglycerides, and the like.

The nutritional composition of the invention may also contain other substances which may have a beneficial effect such as lactoferrin, nucleotides, nucleosides, and the like.

The nutritional composition of the invention may also contain carotenoid(s). In some particular embodiments of the invention, the nutritional composition of the invention does not comprise any carotenoid.

The nutritional composition according to the invention can be for example an infant formula, a starter infant formula, a follow-on or follow-up formula, a baby food, an infant cereal composition, a fortifier or a supplement. In some particular embodiments, the composition of the invention is an infant formula, a fortifier or a supplement that may be intended for the first 4 or 6 months of age. In a preferred embodiment the nutritional composition of the invention is an infant formula.

In some other embodiments the nutritional composition of the present invention is a fortifier. The fortifier can be a breast milk fortifier (e.g. a human milk fortifier) or a formula fortifier such as an infant formula fortifier or a follow-on/follow-up formula fortifier.

In another embodiment, the composition of the invention may be a supplement. The supplement may be in the form of tablets, capsules, pastilles, a powder, a gel or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

Further, the supplement may contain vitamins, minerals trace elements and other micronutrients in accordance with the recommendations of Government bodies such as the USRDA.

When the nutritional composition is a supplement, it can be provided in the form of unit doses. In a specific embodiment the nutritional composition is a supplement in powder form and provided in a sachet. When the supplement is in powder form, it may comprise a carrier. It is however preferred that the supplement is devoid of a carrier. In another embodiment, the supplement is in the form of a syrup. In such case B. longum subsp. longum and LNT are preferably dissolved or suspended in water acidified with citrate.

The nutritional composition according to the invention may be prepared in any suitable manner. A composition will now be described by way of example.

For example, a formula such as an infant formula may be prepared by blending together the protein source, the carbohydrate source and the fat source in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture. LNT may be added at this stage, especially if the final product is to be in liquid form. If the final product is to be a powder, they may likewise be added at this stage if desired.

The liquid mixture is then homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. B. longum subsp. longum is typically added at this stage by dry-mixing. LNT may also or alternatively be added in the same way or by blending in a syrup form of crystals.

If a liquid composition is preferred, the homogenised mixture may be sterilised then aseptically filled into suitable containers or may be first filled into the containers and then retorted.

### Use

The present invention relates to a composition or to a kit of compositions comprising Bifidobacterium longum subsp. longum and Lacto-N-tetraose (LNT) for use in
increasing the metabolic activity of the gut microbiota of the subject consuming the composition, preferably increasing the production of fermentation metabolites, more preferably increasing the production of short chain fatty acids, most preferably increasing the production of acetate and/or propionate; preferably these benefits are achieved in the colon of the subject consuming the composition;
reducing the production of ammonium in the gut, preferably in the colon of the subject consuming the composition;
improving engraftment and/or survival of Bifidobacteria, preferably of Bifidobacterium longum subsp. longum in the colon of the subject consuming the composition;
promoting the growth and/or the survival of beneficial bacteria in the gut and/or the oral cavity of the subject consuming the composition, preferably promoting the growth and/or the survival of Streptococcus salivarius in the gut and/or in the oral cavity, preferably in the colon and/or the oral cavity of the subject consuming the composition;
promoting the growth and/or survival of propionate-producing microorganism in the gut, preferably in the colon of the subject consuming the composition;
treating, preventing and/or reducing inflammation in the subject consuming the composition, preferably inflammatory conditions in the gut and/or in the oral cavity, more preferably in the colon of the subject consuming the composition;
treating, preventing and/or reducing infections, such as bacterial infections, preferably gastrointestinal and/or oral infections (such as pharyngeal infection, for example Streptococcus pyogenes infection) in the subject consuming the composition;
improving immunity in the subject consuming the composition
preventing caries in the subject consuming the composition.

The present inventors have established a synergistic effect of B. longum subsp. longum and LNT leading to increased production of short chain fatty acids and in particular acetate and lactate. The pattern of short chain fatty acids production is an indicator of the microbial carbohydrate metabolism in the colon. The increase in production of acetate and lactate by the combination of B. longum subsp. longum and LNT thus provides evidence of an increased metabolic activity of the microbiota of the subject consuming this synbiotic combination. Short chain fatty acids are further well known to play a crucial role in gut health by promoting carbohydrate metabolism and are related to various health effects, such as against inflammation, against infections and in the improvement of immunity.

Acetate can be used as an energy source for the host and as a potential substrate for lipid synthesis in the body. Moreover, it is an important precursor for the synthesis of butyrate and can exert antimicrobial effects against pathogens. The present inventors have obtained a significant increase of acetate levels after 24 hours incubation with the synbiotic composition of LNT and B. longum subsp. longum.

Levels of propionate have also been synergistically increased by the combination of LNT and B. longum subsp. longum. Propionate acts as the main energy source for the gut epithelium and has shown protective effects against inflammation. Because the administered B. longum subsp. longum is not capable of producing propionate itself, evidence of an increase of propionate levels is evidence of an increase of the propionate-producing members from the background microbiota of the subject consuming the synbiotic composition comprising B. longum subsp. longum and LNT.

A drastic decrease of ammonium production has also been observed by the present inventors as a result of the combination of B. longum subsp. longum and LNT. Ammonium production results from proteolytic microbial activity, which is associated with formation of toxic by-products, such as p-cresol and p-phenol. Ammonium production has been associated directly and indirectly with detrimental health effects. As a consequence, reduction of ammonium production is considered beneficial.

A further beneficial microbial community shift was observed as an effect of the synbiotic combination of B. longum subsp. longum and LNT and resulted in:
the engraftment of B. longum subsp. longum and increased viability of this probiotic
the promotion of the growth of beneficial commensal bacteria, such as Streptococcus 5 salivarius.

The present inventors have provided evidence that the population of B. longum subsp. longum is increased with the synbiotic composition compared to the probiotic alone, whereas no effect was seen with administration of the prebiotic alone. So that it can be concluded that the probiotic B. longum subsp. longum and the prebiotic LNT act synergistically.

The present inventors have also shown that the B. longum subsp. longum and LNT act synergistically to boost the growth of Streptococcus salivarius after 6 and 48 hours of incubation. At both time points after administration, the effect of the combination of B. longum subsp. longum and LNT is larger than the sum of the effects of the probiotic and of the prebiotic alone.

Furthermore, the synergistic effect is particularly effective in improving the survival of S. salivarius. Indeed, the present inventors have shown that the population of this species strongly decreases at 48 hours compared to the population at 6 hours when the probiotic or the prebiotic is administered alone, whereas the population increases with the synbiotic.

Streptococcus salivarius is known to have beneficial health effects, in particular it exhibits anti-inflammatory properties (see for example Kaci et al., Anti-Inflammatory Properties of Streptococcus salivarius, a Commensal Bacterium of the Oral Cavity and Digestive Tract; Applied and Environmental Microbiology 80(3): 928-934), is effective against caries in children (see Burton et al., Influence of the Probiotic Streptococcus Salivarius Strain M18 on Indices of Dental Health in Children: A Randomized Double-Blind, Placebo-Controlled Trial, J. Med Microbiol 2013; 62(6):875-884) and exhibits antimicrobial properties. S. salivarius is an early colonizer of the human oral cavity and gut after birth and is associated with establishment of immune homeostasis and regulation of host inflammatory response, in particular inhibition of inflammation in the colon and in the oral cavity. S. salivarius plays a role against oral infections through the production of anti-microbial peptides. Anti-microbial peptides inhibit for example Streptococcus pyogenes, most important bacterial cause of pharyngeal infection. The nutritional compositions of the present invention are preferably administered orally and therefore B. longum subsp. longum and LNT are deposited in the mouth of the subject when consuming the composition and remain until they are removed for example by the effect of tooth brushing. As shown in the examples the growth of S. salivarius is stimulated early, as shown at 6 hours after administration, so that S. salivarius grown has sufficient time to deploy its effect.

The compositions described herein comprising Bifidobacterium longum subsp. longum and LNT may be for use in the treatment or prevention of inflammation, preferably of gastro intestinal inflammatory conditions.

The gastrointestinal inflammations relate to inflammations involving the gastrointestinal tract. Similarly, there can be inflammations of the upper gastrointestinal tract or of the lower gastrointestinal tract. Examples of gastrointestinal inflammations are enterocolitis and NEC (necrotizing enterocolitis). The gastrointestinal infections may also be associated with a gastrointestinal inflammation.

Enterocolitis is an inflammation of the digestive tract, involving the small intestine and the colon. Common clinical manifestations of enterocolitis are frequent diarrheal defecations, with or without nausea, vomiting, abdominal pain, fever, chills, alteration of general condition. General manifestations are given by the dissemination of the infectious agent or its toxins throughout the body, or most frequently by significant losses of water and minerals, the consequence of diarrhea and vomiting.

Necrotizing enterocolitis (NEC) is a medical condition primarily seen in premature infants, where portions of the bowel undergo necrosis (tissue death). It occurs postnatally and it is the second most common cause of mortality in premature infants. Initial symptoms include feeding intolerance, increased gastric residuals, abdominal distension and bloody stools. The symptoms may progress rapidly to abdominal discoloration with important gut necrosis, intestinal perforation, peritonitis, systemic hypotension requiring intensive medical support, need of a surgical intervention, and sometimes death.

For example the compositions described herein comprising Bifidobacterium longum subsp.

longum and LNT may be for use in the treatment or prevention of a gastro intestinal inflammatory and inflammation associated to a gastro intestinal infections.

The composition comprising Bifidobacterium longum subsp. longum and LNT as described herein are preferably administered enterally. Enteral administration may be oral and/or gastric. In general terms, administration of the composition described herein may, for example, be by an oral route or another route into the gastro-intestinal tract, for example the administration may be by tube feeding.

The nutritional composition according to the invention is for use in infants or young children. The infants or young children may be born term or preterm. In a particular embodiment the nutritional composition of the invention is for use in infants or young children that were born preterm, having a low birth weight and/or born small for gestational age (SGA). In a particular embodiment the nutritional composition of the invention is for use in preterm infants, infants having a low birth weight and/or infants born small for gestational age (SGA).

The nutritional composition of the present invention may also be used in an infant or a young child that was born by C-section or that was vaginally delivered.

In some embodiments the composition according to the invention can be for use before and/or during the weaning period.

The nutritional composition can be administered (or given or fed) at an age and for a period that depends on the needs.

The nutritional composition can be for example given immediately after birth of the infants.

The composition of the invention can also be given during the first week of life of the infant, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the invention, the nutritional composition is given (or administered) to an infant within the first 4, 6 or 12 months of birth of said infant. In some other embodiments, the nutritional composition of the invention is given few days (e.g. 1, 2, 3, 5, 10, 15, 20 . . . ), or few weeks (e.g. 1, 2, 3, 4, 5, 6, 7, 8, 9, 10 . . . ), or few months (e.g. 1, 2, 3, 4, 5, 6, 7, 8, 9, 10 . . . ) after birth. This may be especially the case when the infant is premature, but not necessarily.

In one embodiment the composition of the invention is given to the infant or young child as a supplementary composition to the mother's milk. In some embodiments the infant or young child receives the mother's milk during at least the first 2 weeks, first 1, 2, 4, or 6 months. In one embodiment the nutritional composition of the invention is given to the infant or young child after such period of mother's nutrition, or is given together with such period of mother's milk nutrition. In another embodiment the composition is given to the infant or young child as the sole or primary nutritional composition during at least one period of time, e.g. after the 1^{st}, 2^{nd} or 4^{th} month of life, during at least 1, 2, 4 or 6 months.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1—Investigation of Synbiotic Effect of B. longum Subsp. Longum and LNT

### Aim of the Study

The aim of this project was to evaluate the properties of a synbiotic combination of Bifidobacterium longum subsp. longum ATCC BAA-999 and LNT using short-term colonic incubations (48 h), in which the proximal colon environment of a baby was simulated. The evaluation was based on the effects on microbial metabolic activity (SCFA, lactate and ammonium production) and community composition (16Stargeted Illumina sequencing), through comparison with a blank (no treatment), a probiotic (containing only the B. longum subsp. longum strain) and a prebiotic (containing only LNT).

### Materials and Methods

In these experiments, a simplified simulation of the continuous Simulator of the Human Microbial Ecosystem (SHIME®) was used. This SHIME® model has been extensively used for more than 20 years for both scientific and industrial projects and has been validated with in vivo parameters (see for example Van den Abbeele et al., Arabinoxylo-Oligosaccharides and Inulin Impact Inter-Individual Variation on Microbial Metabolism and Composition, Which Immunomodulates Human cells, J. Agric. Food chem. 2018, 66, 5, 1121-1130). Upon stabilization of the microbial community in the different regions of the colon, a representative microbial community is established in the three colon compartments, which differs both in composition and in functionality in the different colon regions. A single stage batch system mimicking the colonic conditions was used in these experiments as simplified SHIME® system.

Fecal material was collected from five 3-month old baby donors. Fecal suspensions were prepared and mixed with an internally optimized cryoprotectant. The obtained suspensions were aliquoted and preserved at −80° C. (after flash freezing) (cryostock). Preparation of the cryostock from a single fecal suspension ensures that identical microbial communities are obtained in each aliquot, and thus that an identical inoculum is used throughout the different project phases. Moreover, preservation of aliquots ensures that the preserved samples undergo only one freeze-thawing cycle before introduction in a given incubation, as a new aliquot is used for each phase of the project. These actions ensure optimal reproducibility.

Short-term screening assays, as carried out by ProDigest, typically consist of a colonic incubation of a representative dose of the test compound(s) under simulated conditions representative for the proximal large intestine, with a bacterial inoculum obtained from a donor. At the start of the short-term colonic incubation, the test ingredients are added to sugar-depleted nutritional medium containing basal nutrients present in the colon (e.g. host-derived glycans such as mucin).

LNT (1 g/L) and/or Bifidobacterium longum subsp. longum ATCC BAA-999 (inoculated at 1.5×107 CFU/ml at start of the incubation) were added to a sugar-depleted nutritional medium, together with the preserved human baby inoculum. Following incubations were performed (Table 1):
Blank incubations without prebiotic or probiotic (conditions 1-3)
Probiotic incubation, without prebiotics (conditions 4-6 and 13-15)
Prebiotic incubations, without probiotics (conditions 7-9 and 16-18)
Synbiotic incubations (conditions 10-12 and 19-21)

To account for biological variability, all tests were performed in triplicate, resulting in 12 independent incubations. An assessment was made of the SCFA and lactate production at the start of the incubation and after 24 h. Ammonium concentrations were measured at the start and after 48 h of incubation. The effect on the gut microbial community was assessed through Illumina sequencing at the start of the incubation and after 6 h, and 48 h. The methodology applied involves primers that span 2 hypervariable regions (V3-V4) of the 16S rRNA gene. Using a pair-end sequencing approach, sequencing of 2×250 bp results in 424 bp amplicons. Such fragments are taxonomically more informative as compared to smaller fragments. The experiment was performed for 48 h at 37° C., under shaking (90 rpm) and anaerobic conditions. The incubations were performed in fully independent reactors with sufficiently high volume in order to not only ensure robust microbial fermentation, but also to allow the collection of multiple samples over time. Sample collection enables assessment of metabolite production and thus to understand the complex microbial interactions that are taking place. Samples that were analyzed with Illumina sequencing were also analyzed with flow cytometry to determine the number of total bacterial cells, thus allowing to convert the proportional values obtained with Illumina into absolute quantities. Samples were analyzed on a BD Facs verse. The samples were run using the high flow rate. Bacterial cells were separated from medium debris and signal noise by applying a threshold level of 200 on the SYTO channel. Proper parent and daughter gates were set to determine all populations.

TABLE 2
Experimental setup of the experiment

| | B. longum subsp. | | |
|---|---|---|---|
| | longum | LNT | |
| Condition | (CFU/mL) | (g/L) | Replicate |
| 1 | | | A |
| 2 | | | B |
| 3 | | | C |
| 4 | 1.5*10⁷ | | A |
| 5 | | | B |
| 6 | | | C |
| 7 | | 1 | A |
| 8 | | | B |
| 9 | | | C |
| 10 | 1.5*10⁷ | 1 | A |
| 11 | | | B |
| 12 | | | C |

### Results

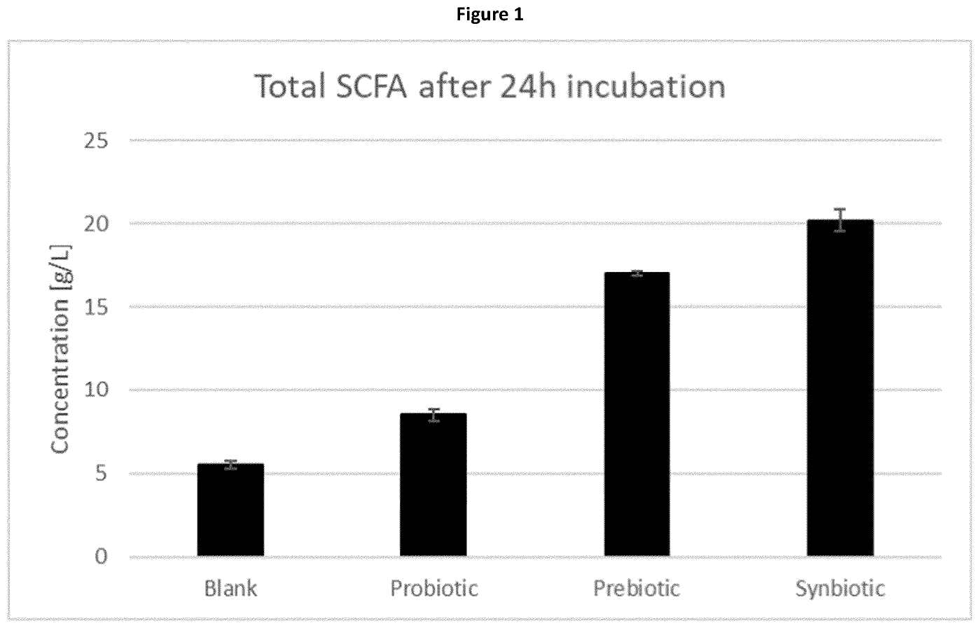

Short chain fatty acids (SCFA) are an assessment of the microbial carbohydrate metabolism and can be compared to typical fermentation patterns for normal GI microbiota. The total SCFA levels are reflective of the overall fermentation of test ingredients. Incubation with the combination of B. longum subsp. longum with LNT resulted in a significant increase of 16% compared to the prebiotic incubation and of 58% compared to the probiotic incubation, after 24 hours of incubation (see FIG. 1).

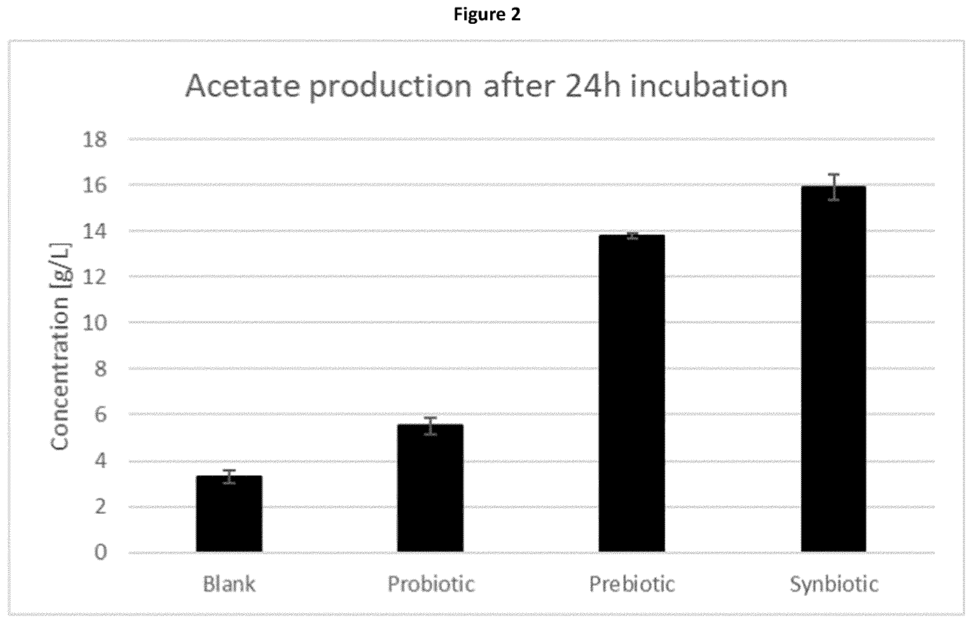

Acetate production was also significantly increased for the incubation with the synbiotic, compared to incubation with the probiotic alone (65%) and with the prebiotic alone (13%). Results are provided in the graph of FIG. 2. Acetate can advantageously be used as an energy source for the host and as a potential substrate for lipid synthesis in the body. Moreover, it is an important precursor for the synthesis of butyrate and can exert antimicrobial effects against pathogens.

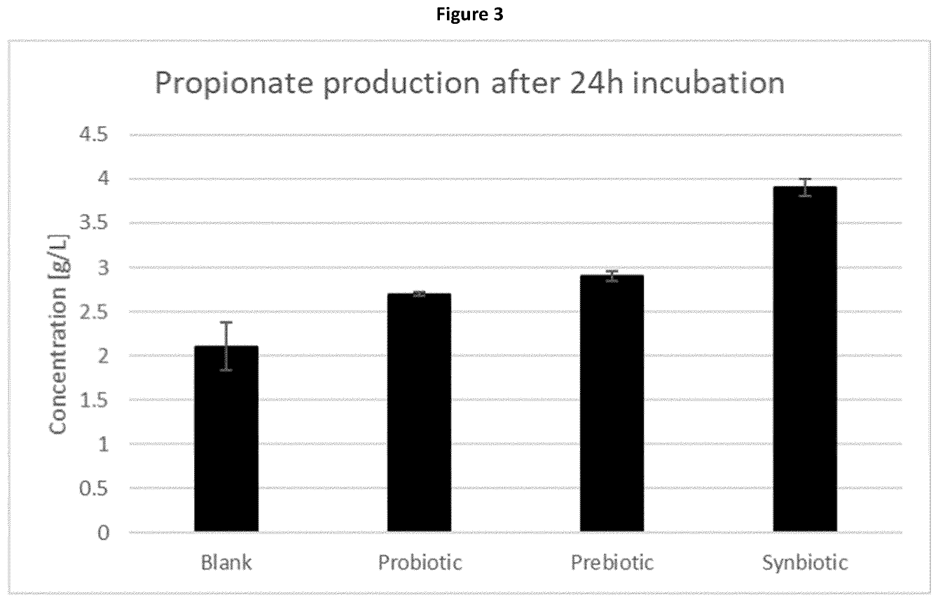

Levels of propionate were also significantly higher for the incubation with the synbiotic composition. The propionate levels measured in the synbiotic incubation were 31% higher than the incubation with the probiotic alone and 26% higher than incubation with the prebiotic alone. The results are presented in the graph of FIG. 3. This result is also an evidence of the efficiency of the synbiotic in promoting propionate-producing members of the background microbiota.

Ammonium production was found to be significantly reduced for the synbiotic incubation in comparison to both the probiotic alone and the prebiotic alone. Ammonium production in the synbiotic incubation was reduced by 97% compared to the blank and to the incubation with the probiotic alone and by 67% compared to the prebiotic alone, after 48 hours of incubation. Surprisingly, the synbiotic has a much stronger effect than the prebiotic, despite the absence of any positive effect of the probiotic alone. Ammonium production is the consequence of the proteolytic microbial activity and is associated with formation of toxic by-products. Ammonium production is associated with adverse health effect and it is therefore desirable to limit ammonium production as much as possible.

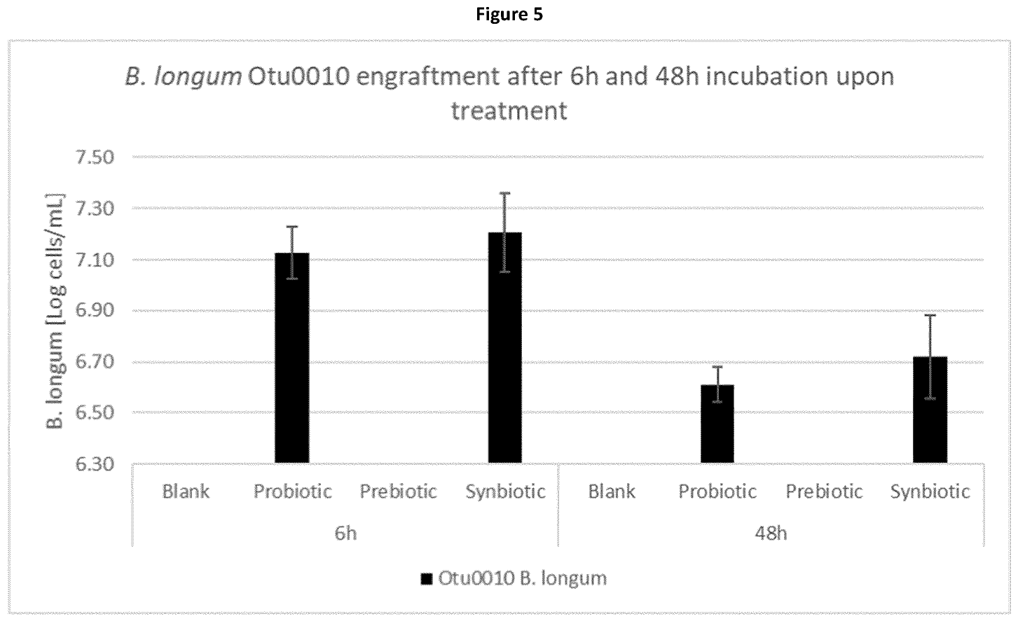

Assessment of the B. longum subsp. longum after 6 and 48 hours of incubation has shown that the population of B. longum subsp. longum is increased by the administration of the synbiotic compared to the probiotic alone, whereas no effect was seen with administration of the prebiotic alone (see FIG. 5). Therefore, it can be concluded that the probiotic B. longum subsp. longum and the prebiotic LNT act synergistically. This is consistently shown at both time points.

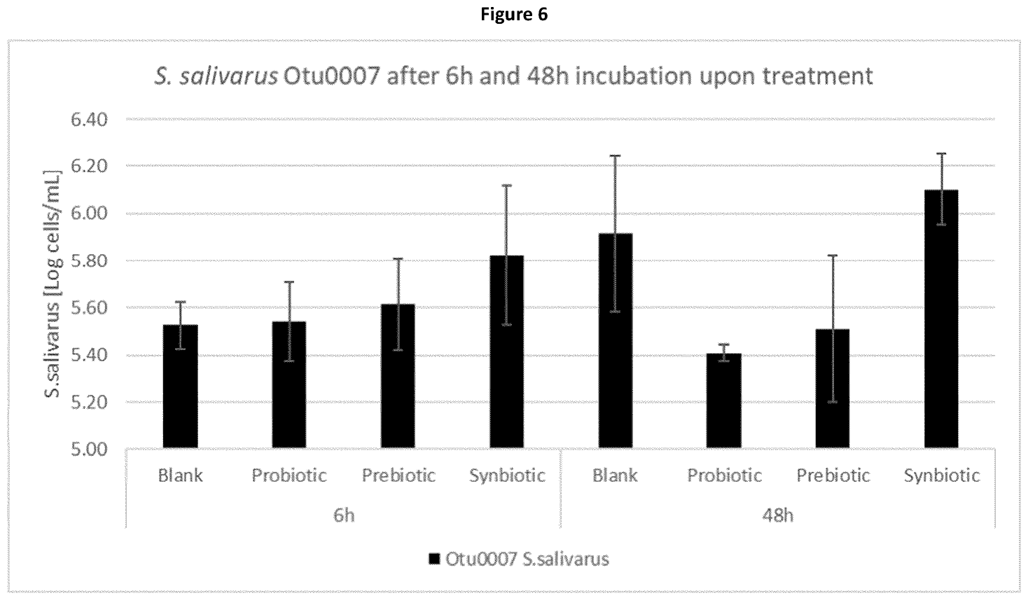

The synbiotic incubation was also found effective in modulating the microbiota population and in boosting the population of beneficial bacteria. For example, the population of the beneficial Streptococcus salivarius is higher in the synbiotic incubation compared to all other incubations. Interestingly, neither the probiotic alone, nor the prebiotic alone were able to boost the population of Streptococcus salivarius after 6 and 48 hours of incubation, whereas the synbiotic achieved respectively a 0.30 and 0.19 log increase of the population of this beneficial OTU compared to the blank incubation. After 6 and 48 hours of incubation the synbiotic incubation provided higher counts of S. salivarius with respectively 0.28 and 0.69 log higher than the probiotic incubation alone. Similar result was obtained when comparing the effect of synbiotic incubation with prebiotic alone. 0.21 log and 0.59 log increase of the OTU Streptococcus salivarius was obtained after 6 hours and 48 hours of incubation, respectively. Such increase of the population of S. salivarius is beneficial due to the known benefits of this species, namely its anti-inflammatory properties. The results are provided in FIG. 6.

1. A method for achieving a result from the group consisting of:
a. increasing the metabolic activity of the gut microbiota of the subject consuming the composition,
b. reducing the production of ammonium in the gut of the subject consuming the composition;
c. improving engraftment and/or survival of Bifidobacteria in the colon of the subject consuming the composition;
d. promoting the growth and/or the survival of beneficial bacteria in the gut and/or in the oral cavity of the subject consuming the composition;
e. promoting the growth and/or survival of propionate-producing microorganism in the gut of the subject consuming the composition;
f. treating, preventing and/or reducing inflammation in the subject consuming the composition;
g. treating, preventing and/or reducing infection in the subject consuming the composition;
h. improving immunity in the subject consuming the composition; and/or
i. preventing caries in the subject consuming the composition;
comprising administering LNT and B. longum subsp. longum to a subject, wherein the subject is an infant (up to 12 months of age) or a young child aged from 1 to 3 years of age.

2. A method according to claim 1, for use in increasing the production of fermentation metabolites in the colon of the subject.

3. A according to claim 1, for use in increasing the production of short chain fatty acids, such as acetate and propionate, in the colon of the subject.

4. A method according to claim 1, for use in improving engraftment and/or survival of Bifidobacterium longum subsp. longum.

5. A method according to claim 1, for use in promoting the growth and/or the survival of Streptococcus salivarius in the gut and/or in the oral cavity of the subject consuming the composition.

6. A method according to claim 1, for use in treating, reducing and/or preventing gastrointestinal and/or oral inflammation in the subject.

7. A method according to claim 2, wherein the gastrointestinal inflammation is selected from enterocolitis and NEC (necrotizing enterocolitis).

8. A method according to claim 1, for use in treating, reducing and/or preventing gastrointestinal and/or oral infection in the subject.

9. A method according to claim 1, wherein the infection is a bacterial infection.

10. A method according to claim 1, wherein the infection is a pharyngeal infection.

11. A method according to claim 1, wherein the Bifidobacterium longum subsp. longum is selected from the group consisting of Bifidobacterium longum subsp. longum ATCC BAA-999, Bifidobacterium longum subsp. longum strain CNCM I-2169, Bifidobacterium longum subsp. longum strain CNCM I-2171, Bifidobacterium longum subsp. longum strain ATCC 15708, Bifidobacterium longum subsp. longum strain DSM 20097, Bifidobacterium longum subsp. longum strain NCIMB 8809, Bifidobacterium longum subsp. longum strain CNCM I-2618, Bifidobacterium longum subsp. longum strain CNCM I-2170, Bifidobacterium longum subsp. longum strain ATCC 15707 (T), Bifidobacterium longum subsp. longum strain CNCM I-103, Bifidobacterium longum subsp. longum strain CNCM I-2334, Bifidobacterium longum subsp longum strain CNCM I-3864, Bifidobacterium longum subsp longum strain CNCM I-3853, of and a combination thereof.

12. A method according to claim 1, wherein the Bifidobacterium longum subsp. longum is B. longum subsp. longum ATCC BAA-999.

13. A method according to claim 1, wherein the B. longum subsp. longum is produced by a method of growing the B. longum subsp. longum in a culture medium comprising LNT.

14. A method according to claim 1, wherein the medium comprises LNT at a concentration of 0.02 to 2 wt %.

15. A method according to claim 1, comprising between 10³ to 10¹² cfu of Bifidobacterium longum subsp. longum per daily dose.

16. A method according to claim 1, wherein LNT is present in an amount of 1-5 g/L of the composition.

17. A method according to claim 1, wherein the nutritional composition is in a form selected from the group consisting of an infant formula, a starter infant formula, a follow-on or follow-up formula, a baby food, an infant cereal composition, a fortifier and a supplement.

18. A method for achieving a result selected from the group consisting of:
a. increasing the metabolic activity of the gut microbiota of the subject consuming the composition,
b. reducing the production of ammonium in the gut of the subject consuming the composition;
c. improving engraftment and/or survival of Bifidobacteria in the colon of the subject consuming the composition;
d. promoting the growth and/or the survival of beneficial bacteria in the gut and/or in the oral cavity of the subject consuming the composition;
e. promoting the growth and/or survival of propionate-producing microorganism in the gut of the subject consuming the composition;
f. treating, preventing and/or reducing inflammation in the subject consuming the composition;
g. treating, preventing and/or reducing infection in the subject consuming the composition;
h. improving immunity in the subject consuming the composition; and/or
i. preventing caries in the subject consuming the composition;
comprising administering to a subject a growing-up milk comprising LNT and B. longum subsp. longum, wherein the subject is a child above 3 years and below 8 years of age.
